(19) United States
(12) Patent Application Publication
(10) Pub. No.: US 2007/0238661 A1
(43) Pub. Date: Oct. 11, 2007

(54) Substance P treatment for hepatitis C

(76) Inventor: Mark Lee Witten, (US)

Correspondence Address: Mark L. Witten, Ph. D., 7032 E. Rosewood St., Tucson, AZ, 85710-1236, US

(21) Appl. No.: 11/705,184

(22) Filed: Feb. 12, 2007

Related U.S. Application Data
(60) Provisional application No. 60/790,753, filed on Apr. 11, 2006.

(51) Int. Cl. A61K 38/17

(52) U.S. Cl. 514/12

(57) ABSTRACT

Substance P treatment has been demonstrated to be efficacious in a human Hepatitis C patient. Fibrosis of the liver is very damaging to long-term survival with Hepatitis C disease. Substance P treatment also has been demonstrated to preserve fibrinogen levels in the body.

### BACKGROUND OF THE INVENTION

Substance P belongs to a family of bioactive neuropeptides known as the tachykinins. Payan (1989) Ann. Rev. Med. 40:341. We have discovered that substance P has anti-viral actions with a Hong Kong influenza respiratory virus (Witten, et al., OIE/FAO International Scientific Conference on Avian Influenza, Dev Biol (Basel). 124:259 (2006)). We have also discovered in an acute lung injury model that a substance P analog, Sar⁹, Met (O₂)¹¹-substance P, demonstrates the ability to preserve fibrinogen concentrations in the lungs after acute formalin exposure by proteomic analyses. Fibrosis is a serious condition caused by the Hepatitis C virus (Koda, et al., Hepatology 45:297-306 (2007)). At the present time, there is no known cure for Hepatitis C (Salmeron, et al., J Viral Hepat 14: 89-95 (2007)).

### SUMMARY OF THE INVENTION

It is an object of the invention to provide a method of regulating an immune response to a viral infection, such as Hepatitis C.

It is still another object of the invention to provide a method of decreasing viral titers of Hepatitis C by direct action of substance P on the virus.

It is yet another object of the invention to utilize substance P in combination with standard hepatitis C treatment of ribavirin and interferon, alpha.

It is yet another object of the invention to prevent the development of fibrosis in the liver of the Hepatitis C patients.

These and other objects of the invention are provided by one or more of the embodiments described below. In one embodiment of the invention a method of regulating the immune system of a hepatitis C patient is provided. The method comprises the steps of:

administering an immune-regulating amount of aerosolized substance P, or a bioactive analogue thereof, to a patient infected with Hepatitis C virus.

According to another embodiment of the invention, a method of stimulating the proliferation of immune cells in invitro cell culture is provided.

The method comprises:

utilizing a mixed immune cell culture with substance P, or a bioactive analogue thereof, in an amount effective to stimulate the proliferation of the immune cells in culture for re-injection back into the body of the Hepatitis C patient during anti-viral treatment.

Yet another embodiment of the invention, a method of direct inhibition of the Hepatitis C virus is provided. The method comprises the steps of: administering an effective amount of substance P, or a bioactive analogue thereof, to a patient infected with Hepatitis C to cause a decrease in viral replication or a direct action of inhibition or killing of the hepatitis C virus.

Another embodiment of the invention, a method of combining substance P therapy with standard hepatitis C treatment of ribavirin and interferon alpha is provided. The method comprises the steps of administering an effective amount of substance P, or a bioactive analogue thereof, to a patient infected with Hepatitis C in combination with standard drug treatment of ribavirin and interferon alpha.

Finally, yet another embodiment of the invention, a method of direct inhibition of fibrosis of the Hepatitis C-infected liver is provided. The method comprises the steps of administering an effective amount of substance P, or a bioactive analogue thereof, to a patient infected with Hepatitis C to cause an inhibition of fibrosis in the liver by preservation of fibrinogen levels.

These and other embodiments which are described in more detail below, provide the art with prophylactic and therapeutic means of treatment for Hepatitis C.

### DETAILED DESCRIPTION OF THE PREFERRED EMBODIMENTS

We have determined that Substance P, a neuropeptide, also has anti-viral properties against Hong Kong influenza virus and hepatitis C virus and preserves lung fibrinogen levels. This has been determined in three different model systems. In one model system, the deleterious effects of Hong Kong influenza respiratory virus was reversed and/or prevented by Substance P aerosol therapy. A/Hong Kong 8/68 influenza virus was used in a murine model of occupational toxicology inducing acute lung injury. C57BL/6 mice were exposed to jet fuel inhalation one hour/day over seven days to attenuate the pulmonary immune response. At day +7, mice received the A/Hong Kong 8/68 influenza virus by nasal inoculation. The mice were divided into two groups: A/Hong Kong 8/68 virus only and mice receiving Sar⁹, Met (O₂)¹¹-substance P, one microMolar/15 minutes/day via aerosol inhalation. A/Hong Kong 8/68 virus only developed influenza symptoms of decreased body mass, fever, dehydration, nasal discharge after five days and at Day +7 began to die of acute lung injury. The Sar⁹, Met (O₂)¹¹-substance P-treated mice did not develop influenza symptoms, however, they were killed at Day +7 to compare lung tissues between the two groups of mice. The Sar⁹, Met (O₂)¹¹-substance P-treated mice showed leukotriene B₄ levels at 33% of the A/Hong Kong 8/68 virus only mice and normal lung cell counts after broncho-alveolar lavage procedures. Electron micrographs of A/Hong Kong 8/68 virus only mice showed absence of airway cilia, swollen airway epithelial cells, large numbers of mitochondria, and colonies of A/Hong Kong 8/68 virus. We did not observe any signs of lung injury in the Sar⁹, Met (O₂)¹¹-substance P-treated mice infected with A/Hong Kong 8/68 virus.

In a second system, a human patient infected with Hepatitis C virus, was treated under compassionate use in collaboration with his personal physician, with substance P aerosol therapy for sixty days. This patient demonstrated a 10-fold reduction in Hepatitis C viral counts from 1.3 million viral copies/ml to 130,000 viral copies/ml. Additionally, under the patient's previous standard drug treatment of ribavirin and interferon alpha for his Hepatitis C disease, he would typically suffer a loss of apatite, nausea, vomiting, and significant weight loss of about 20% of body mass. These deleterious effects were eliminated with substance P aerosol treatment combined with his standard drug treatment of ribavirin and interferon alpha.

In a third system, we sought to determine if Sar⁹, Met (O₂)¹¹-substance P-treated Fischer 344 rats who were exposed to 67 mg/m³ (67 ppm) aerosolize formalin for 1.5 minutes would attenuate lung protein alterations. At approximately 42 minutes post-formalin or air exposure, rat lungs were removed for protein analysis. Two-dimensional gel electrophoresis revealed altered abundance of 71 proteins whose formalin-mediated changes were attenuated in the Sar⁹, Met (O₂)¹¹-substance P-treated formalin-exposed rats' lungs. Among these 71 proteins were fibrinogen-related proteins.

Substance P (RPKPQQFFGLM-NH₂) or any of its bioactive analogues can be used in the methods of the present invention. These include, but are not limited to: [Met-OH¹¹]-substance P, [Met-OMe¹¹]-substance P, [Nle¹¹]-substance P, [Pro⁹]-substance P, [Sar¹¹]-substance P, [Tyr⁸]-substance P, [p-Cl-Phe^{7,8}]-substance P, and Sar⁹, Met (O₂)¹¹-substance P. The latter analogue is particularly preferred. Bioactive analogs, according to the invention are those which act as competitive inhibitors of substance P by binding to the SP receptors (NK-1, NK-2, or NK-3 receptors). Other derivatives as are known in the art and commercially available (e.g., from Sigma, CS-BIO, or PolyPeptide Laboratories) can be used. In addition, substance P fragments and derivatized substance P fragments may also be used. Substitution, deletion, or insertion of one to eight amino acid residues, and preferably from one to three amino acid residues, will lead to analogs which can be routinely tested for biological activity. In addition, functional groups may be modified on substance P while retaining the same carbon backbone. Again, routine testing will determine which of such modifications do not adversely affect biological activity.

Typical concentration ranges of substance P or its bioactive analogues in the aerosol administered is between 0.0001 and 75 microMolar. Concentrations in the range of between 0.05 and 50 microMolar are particularly useful. In the case of administration of a mixed immune system cell culture, the substance P or its bioactive analogue need not be aerosolized. It can be easily administered as a liquid at a concentration between about 0.0001 and 75 microMolar.

Among the subjects for whom aerosolized substance P therapy will be useful are those with Hepatitis C. Suitable devices for administering the aerosol of the present invention include nebulizers as well as hand-held “puffer” devices typically used in asthmatic patients at the present time.

Suitable treatment regimens for treatment according to the present invention include daily treatment by aerosol. Other modes of treatment include continual transdermal infusion, intravenous injection, subcutaneous injection, mixed in a gel and applied for absorption into the skin, and orally. Suitable formulations of substance P for administration are any which are pharmaceutically acceptable and in which substance P retains its biological activity. Generally, such formulations are substance P dissolved in normal sterile (0.09% sodium chloride) saline.

We claim:

1. A method of stimulating the immune system of a Hepatitis C patient, comprising the steps of:
administering an immune-regulatory amount of aerosolized substance P, or a bioactive analogue thereof, to a person infected with Hepatitis C.

2. The method of claim 1 wherein a bioactive analogue is administered.

3. The method of claim 2 wherein the bioactive analogue is selected from the group consisting of: [Met-OH¹¹]-substance P, [Met-OMe¹¹]-substance P, [Nle¹¹]-substance P, [Pro⁹]-substance P, [Sar⁹]-substance P, [Tyr⁸]-substance P, [p-Cl-Phe^{7,8}]-substance P, and Sar⁹, Met (O₂)¹¹-substance P.

4. The method of claim 3 wherein the bioactive analogue is [Sar⁹, Met (O₂)¹¹-substance P.

5. The method of claim 1 wherein the concentration of substance P or the bioactive analogue in the administered aerosol or intra-venous is between 0.0001 and 75 microMolar concentration.

6. A method of stimulating invitro immune system cells comprising:
contacting a mixed invitro immune system cell culture with substance P, or a bioactive analogue thereof, in a concentration found effective in stimulating these cells for injection into the body of a Hepatitis C patient.

7. The method of claim 6 wherein the amount of substance P or bioactive analogue is between 0.0001 and 75 microMolar concentration.

8. A method of direct inhibition of the Hepatitis C virus in a patient by attenuating viral replication or a direct action of inhibition or killing of the Hepatitis C virus comprising:
administering an immune-regulating amount of aerosolized or intra-venous substance P, or a bioactive analogue thereof, to a Hepatitis C patient.

9. A method of combining substance P therapy with standard Hepatitis C treatment of ribavirin and interferon alpha is provided. The method comprises:
administering an effective amount of substance P, or its bioactive analogue thereof, to a patient infected with Hepatitis C in combination with standard drug therapy of ribavirin and interferon alpha.

10. A method of direct inhibition of fibrosis of the Hepatitis C-infected liver by administering an effective amount of substance P, or a bioactive analogue thereof, to a patient infected with Hepatitis C to cause an inhibition of fibrosis in the liver by preservation of fibrinogen levels.

11. The method of claim 1, 6, 8, 9, or 10 wherein the route of administration is by inhalation.

12. The method of claim 11 wherein the administration is intranasal.

13. The method of claim 11 wherein the administration is oral.

14. The method of claim 11 wherein the administration is intra-venous.

15. The method of claim 11 wherein the administration is transdermal infusion.

16. The method of claim 11 wherein the administration is sub-cutaneous injection.

17. The method of claim 11 wherein the administration is mixed in a gel and applied for absorption into the skin.
